(19) United States
(12) Patent Application Publication
(10) Pub. No.: US 2008/0281145 A1
(43) Pub. Date: Nov. 13, 2008

(54) Golgi Stimulator

(76) Inventors: Issam Nemeh, Westlake, OH (US); George Nicholas Szatkowski, Yorktown, VA (US)

Correspondence Address: George Szatkowski, 210 Edgehill Lane, Yorktown, VA, 23692, US

(21) Appl. No.: 11/746,634

(22) Filed: May 9, 2007

(51) Int. Cl. A61N 1/00

(52) U.S. Cl. 600/13

(57) ABSTRACT

Method and apparatus to improve blood circulation, increase muscle and bone mass, improve joint tissue vitality, repair damaged nerves, and reduce scar tissue, by inducing the targeted biological muscle tissue with a low frequency, northerly polarized, pulsed, DC magnetic field concurrently with a low frequency vibration applied directly to the body's muscle tissue to stimulate the Golgi tendon organs by repetitive displacement of the targeted muscle tissue.

Golgi tendon organs are located in the collagen fibers of the tendon where the tendon and muscle fibers merge. Like muscle spindles, Golgi tendons measure changes in the muscle. Although muscle spindles are active even while the muscle is at rest, Golgi tendon organs are only activated when the tendon contracts. Golgi tendon organs are not affected by changes in muscle length, but by the increased tension of the muscle as a result of a change in its length. Our invention patents the idea that Golgi tendons can be artificially stimulated without muscle flexing or physical exercise by applying low frequency pulsed DC magnetic fields in conjunction with repetitive muscle tissue displacement (vibration) at the skin surface of the targeted muscle region. The brain responds to the signals sent from the stimulated Golgi tendons by increasing the blood flow to the bones and muscles in that region. Using this technique to artificially stimulate the Golgi tendons will create improved blood circulation and joint tissue vitality along with increased muscle and bone mass. The invention encompasses an apparatus that delivers stimuli of both magnetic field and vibration to the body's Golgi tendon organs. Golgi tendon organs can be stimulated into triggering by applying low frequency, north polarized, pulsed DC magnetic fields with field strength in the range of 0.0005 to 23 gauss pulsed at 1 to 200 Hz in conjunction with a vibration force of sufficient amplitude to accelerate the targeted muscle tissue in the range of 0.3 to 40 meters per second squared acceleration accompanied by displacement of the muscle tissue by approximately a quarter inch to one inch, at a frequency of 1 to 150 Hz. These stimuli are delivered using stationary or portable applicator pads applied directly to the skin surface to cause muscle tissue displacement.

References Cited
US Patent Documents

| | | | |
|---|---|---|---|
| 5669868 | September, 1997 | Markoll | 600/14 |
| 6123658 | September, 2000 | Schweighofer, et al. | 600/13 |
| 6149577 | November, 2000 | Bouldin, et al. | 600/13 |
| 6899667 | May, 2005 | Becker, et al. | 600/9 |

### DESCRIPTION

1. Field of the Invention

This invention has a multitude of uses in the treatment of human and animal health conditions and general well being, including but not limited to an increase of muscle and bone mass, improvement of joint tissue vitality and blood circulation, repairing damaged nerves, and reducing scar tissue. These health benefits are achieved by inducing the targeted biological muscle tissue with a low frequency northerly polarized pulsed DC magnetic field concurrently with a low frequency vibration applied directly to the body surface to cause repetitive muscle tissue displacement.

2. Description of Prior Art

Interest and practice of magnetotherapy has increased in recent years for a wide variety of physical health issues that include: severe pain management, cancer treatment, mental and neurological disorders including depression, Multiple Sclerosis and Parkinson Disease. A wide spectrum of magnetotherapy patents abounds in both US and foreign patent libraries. The simplest devices are comprised of micro gauss permanent DC magnets mounted in a bracelet or blanket to apply a small static magnetic field to the body for pain reduction. More sophisticated systems encompass elaborate pulsed AC shaped waveforms delivered through multiple coils mounted in a helmet to treat patients for depression. No prior patent has defined the benefit of the additional stimulus of simultaneously adding vibration with the magnetic field to compound the health benefits.

### SUMMARY OF THE INVENTION

Golgi tendon organs are located in the collagen fibers of the tendon where the tendon and muscle fibers merge. Like muscle spindles, Golgi tendons measure changes in the muscle. Although muscle spindles are active even while the muscle is at rest, Golgi tendon organs are only activated when the tendon contracts. Golgi tendon organs are not affected by changes in muscle length, but by the increased tension of the muscle as a result of a change in its length. Our invention patents the idea that Golgi tendons can be artificially stimulated without muscle flexing or physical exercise by applying low frequency pulsed DC magnetic fields in conjunction with repetitive muscle tissue displacement (vibration) at the skin surface of the targeted muscle region. The brain responds to the signals sent from the stimulated Golgi tendons by increasing the blood flow to the bones and muscles in that region. The key points involved in successfully stimulating the Golgi tendon is to apply repetitive displacement of the muscle tissue to create a stretch in the muscle in conjunction with a dc pulsed magnetic field radiated in the same region. Golgi receptors can be artificially stimulated without muscle flexing by applying low frequency pulsed magnetic fields and repetitive skin pressure (vibration) at the surface of the skin in the region of the targeted muscle. This invention can produce the appropriate level of low frequency pulsed magnetic field and vibration to stimulate the Golgi tendons to request more blood flow to the region. The increased blood flow in conjunction with yet other unknown biological and molecular manifestations from these stimuli provide the following health benefits:
1. Increased muscle and bone mass without exercise
2. Improved joint tissue vitality
3. Improved blood circulation
4. Repaired nerve damage
5. Reduced scar tissue
6. Blood vessel dilation
7. Other health benefits

### DRAWINGS

### Brief Description

The assembly and construction of the invention will become clear from the text description and drawing presented here, all of which disclose a non-limiting form of the invention. Many variations to this apparatus can be explored, but the simplest technique to construct the Golgi stimulator will be shown here.

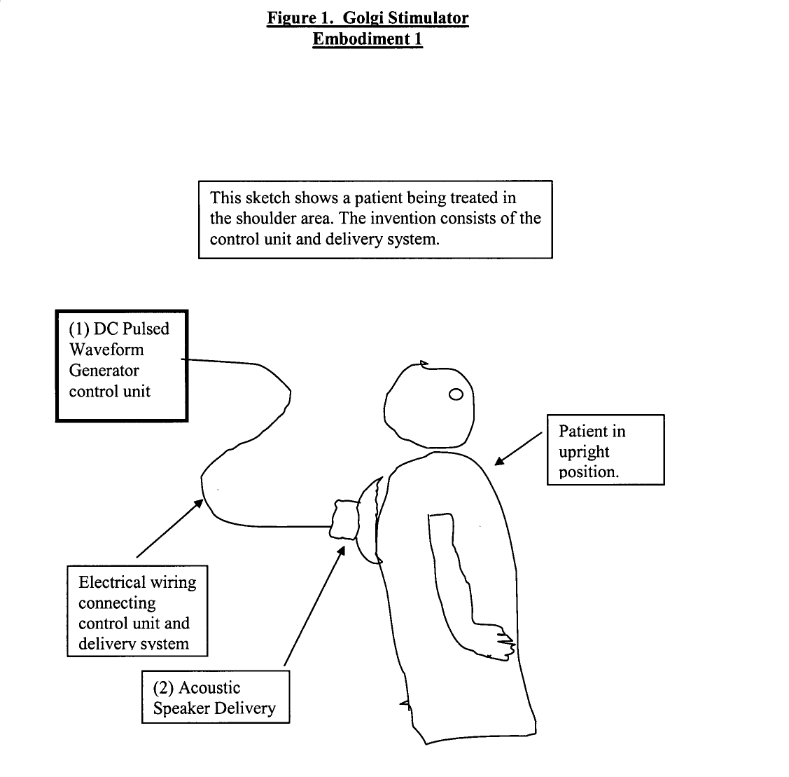

FIG. 1 describes a simple embodiment of the Golgi stimulator that can be assembled using off the shelf components. This design uses a waveform generator control unit (1) with two independent output signals, one square wave and the other a sine wave. The Tenma Company manufactures an inexpensive Sine/Square Wave Generator, Model 72-490, which can work for this purpose. The waveform generator output waveforms share a common frequency control that functions over most of the specified frequency operating parameters as described in the invention claims in addition to having independent amplitude control. The square wave output signal is fed into an audio amplifier (2) to boost the signal to around 40 Volts pulsed DC before it is delivered to the electrical coil (3) to generate the magnetic field stimulus. The square pulsed waveform has sharp rise and fall times which are required to cause the rapid current change in the coil to induce the desired magnetic fields. A lower duty cycle DC pulse could be used in place of the square wave if an appropriate waveform generator were available. A square wave will cause the coil wire to heat more significantly than a lesser duty cycled DC pulse signal, but if the coil wire is sized appropriately over heating will not be an issue. The audio amplifier (2) used to boost the square wave voltage is a common stereo component that provides amplification to alternate input signals, such as an external CD, VCR or phonograph to play through the sound system speakers. A microphone amplifier is another off the shelf component that could be used for this purpose. In our simple embodiment, we used two Pioneer stereo receivers, model 5X-355. One amplifier was used to boost the square wave, the other provided amplification to the sine wave to drive the acoustic speaker to provide the mechanical vibration for tissue displacement. For our purposes, we used an off-the-shelf sub woofer acoustic speaker as our mechanical vibration device (4). We choose an 8 inch diameter sub woofer speaker with maximum power output of 150 Watts. In this embodiment, the sine wave is used to drive the vibration stimulus to reduce the unwanted speaker audio. The coil was made from 112 turns of 20 gauge magnetic coil wire wound around a 2 inch diameter air core. The inductance was calculated to be about 10 millihenries.

The device is used by resting a patient's targeted body tissue on the delivery system and turning on the device. A pulsed DC magnetic field and vibration are delivered to the body tissue inducing artificial stimulation of the patient's Golgi receptors. This simplified embodiment possesses the ability to produce both the specified simultaneous magnetic field and vibration applied to the surface of the patient's skin.

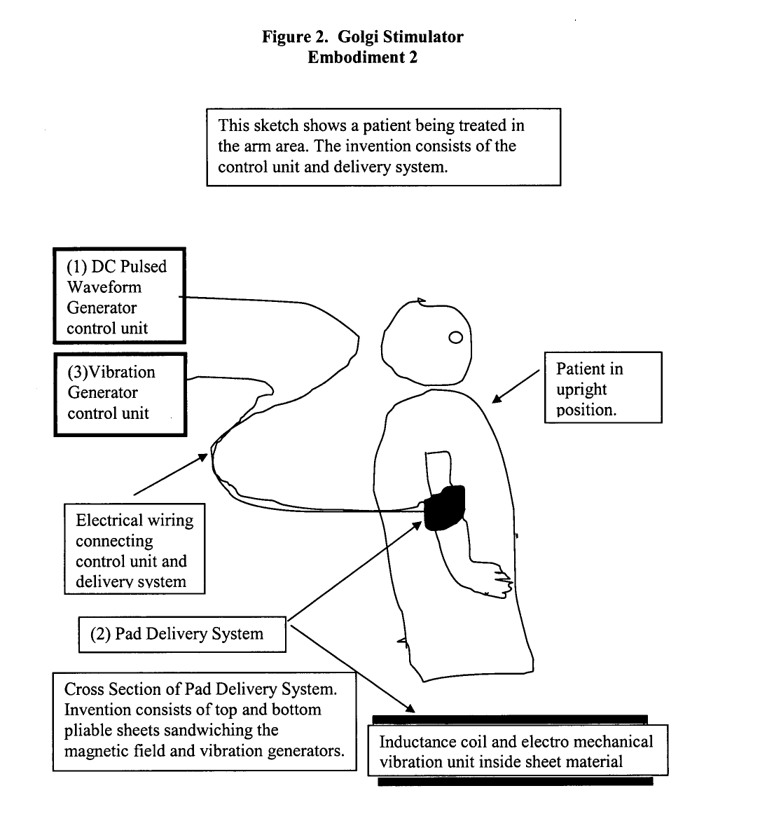

FIG. 2 describes a second embodiment for this invention. In this embodiment, separate controls are utilized to operate the magnetic field and vibration stimuli. The device contains a pulsed DC waveform generator (1) to drive inductance coils which can deliver a magnetic field of specified characteristics as described in the invention claims. The output signal is connected to a pad delivery system (2) containing one or more inductance coils of proper size(s) and shape(s) to deliver the required magnetic field as described in the claims. The inductance coils can be housed behind a sheet of pliable material such as a durable medical grade vinyl or rubber similar to the pressure arm bands used for monitoring blood pressure or the material used in heating pad applications. The inductance coils are spaced around the band to allow the operational device to induce total magnetic field coverage over the wrapped targeted body tissue. The pad delivery system can be positioned on the body in various configuration methods. One method is to incorporate the pad in a device similar to a blood pressure arm band that is strapped around the arm to wrap the bicep. One purpose of this embodiment could be to induce blood vessel dilation for ease in collecting blood samples. The pad delivery system can be configured in a variety of ways to apply the artificial stimuli to treat other body areas. It can be contoured, integrated, and/or upholstered into surfaces such as; clothing or blankets that snuggly contact the skin, keyboard wrist cushions for typists or computer users, elbow resting table tops used in collecting blood samples, beds, or lounge chairs to provide the patient direct contact with the pad. Treatment is achieved with the patient working or resting with the invention operating.

In conjunction with the inductance coils, a unit to create vibration must also be included. This can be achieved by incorporating various types of electromechanical devices found in commercially available massaging units, children's vibrating toys, or simple solenoids appropriately modified to fit within the confines of the delivery system. The vibration unit must operate under its own variable control unit (3) that supplies the desired signals to the electromechanical vibration device to produce the required stimulus described in the claims. For best results, separate vibration and magnetic field control is required to provide optimization of both frequency and amplitude of both the magnetic field and vibration parameters.

1. A method of patient treatment to stimulate the Golgi receptors to trigger responses in the central nervous system providing health benefits that include the following: increased muscle strength and stamina without exercise, improved blood circulation and increased blood vessel dilation, rebuilding of bone mass, reduced scar tissue, and increased nerve regeneration; the treatment is delivered through the application of stimuli comprised of: (a) a DC pulsed magnetic field with amplitude between 0.0005 to 23 gauss in the frequency range of 1 to 200 Hz, with the north polarized field directed toward the patient. The magnetic field is generated by a circular, elliptical or linear wound coil or permanent magnet combination of sufficient size to produce a 4 to 6 inch diameter magnetic flux field to penetrate the desired field level a minimum of 2 to 3 inches into the targeted body tissue and (b) a vibration at the surface of the targeted body tissue in the range of 0.3 to 40 meters per second squared acceleration accompanied by displacement of the muscle tissue by approximately a quarter inch to one inch, at a frequency of 1 to 150 Hz with the stimuli delivered daily for 10 to 45 minutes.
